(19) United States
(12) Patent Application Publication
(10) Pub. No.: US 2007/0093894 A1
(43) Pub. Date: Apr. 26, 2007

(54) Incorporation of antimicrobial combinations onto devices to reduce infection

(75) Inventor: Rabih O. Darouiche, Houston, TX (US)

Correspondence Address: DALLAS OFFICE OF FULBRIGHT & JAWORSKI L.L.P., 2200 ROSS AVENUE, SUITE 2800, DALLAS, TX, 75201-2784, US

(73) Assignees: Baylor College of Medicine, Houston, TX (US); Gov. of the USA As Represented by the Secretary of the Dept. of Health and Human Services, Rockville, MD (US)

(21) Appl. No.: 11/586,407

(22) Filed: Oct. 25, 2006

Related U.S. Application Data
(60) Provisional application No. 60/729,826, filed on Oct. 25, 2005.

(51) Int. Cl. A61F 2/02; A61L 33/00; B05D 3/00

(52) U.S. Cl. 623/11.11; 427/2.1; 427/2.24; 604/265

(57) ABSTRACT

This invention relates to a method for coating a medical device comprising the steps of applying to at least a portion of the surface of the medical device, a bactericidal coating layer, wherein the bactericidal coating layer comprises a bactericidal agent; and applying to at least a portion of the surface of the medical device, a bacteriostatic coating, wherein the bacteriostatic coating layer comprises a bacteriostatic agent wherein the combination of the bactericidal and bacteriostatic agents are in an effective concentration to inhibit growth of microbial organisms relative to an uncoated medical device. The two antimicrobial agents are used to develop a kit comprising these compositions in one container or in separate containers. The kit is used to coat or flush medical devices prior to or after implantation in a mammal.

### CROSS-REFERENCE TO RELATED APPLICATIONS

This application claims priority to U.S. Provisional Application No. 60/729,826 filed Oct. 25, 2005, which is incorporated herein by reference in its entirety.

### STATEMENT REGARDING FEDERALLY SPONSORED RESEARCH OR DEVELOPMENT

The United States Government may have certain rights in this application.

### TECHNICAL FIELD

This invention relates to the field of medicine. More particularly, it relates to medical devices and the combination of antibiotic compositions to coat and/or flush medical devices to decrease or reduce microbial infections and/or growth.

### BACKGROUND OF THE INVENTION

A. Medical Implants

Colonization of bacteria on the surfaces of the implant or other part of the device can produce serious patient problems, including the need to remove and/or replace the implanted device and to vigorously treat secondary infective conditions. A considerable amount of attention and study has been directed toward preventing such colonization by the use of antimicrobial agents, such as antibiotics, bound to the surface of the materials employed in such devices.

Various methods have previously been employed to contact or coat the surfaces of medical devices with an antimicrobial agent. For example, one method would be to flush the surfaces of the device with an antimicrobial containing solution, see e.g. U.S. Pat. No. 6,719,991.

A known method of coating the devices is to first apply or absorb to the surface of the medical device a layer of tridodecylmethyl ammonium chloride (TDMAC) surfacant followed by an antiobiotic coating layer, see e.g. U.S. Pat. No. 6,719,991.

Another successful coating method is impregnation of an antimicrobial agent. The antimicrobial agent penetrates and is incorporated in the exposed surfaces. The antimicrobial composition is formed by dissolving an antimicrobial agent in an organic solvent, adding a penetrating agent, and adding an alkalinizing agent to the composition. See, e.g., U.S. Pat. No. 5,902,283 and U.S. Pat. No. 5,624,704.

A further method known to coat the surface of medical devices with antiobiotics involves first coating the selected surfaces with benzalkonium chloride followed by ionic bonding of the antiobiotic composition. See, e.g., Solomon, D. D. and Sherertz, R. J., J. Controlled Release, 6:343-352 (1987) and U.S. Pat. No. 4,442,133.

These and many other methods of coating medical devices with antibiotics appear in numerous patents and medical journal articles. Other methods of coating surfaces of medical devices with antibiotics are disclosed in U.S. Pat. No. 4,895,566 (a medical device substrate carrying a negatively charged group having a pKa of less than 6 and a cationic antibiotic bound to the negatively charged group); U.S. Pat. No. 4,917,686 (antibiotics are dissolved in a swelling agent which is absorbed into the matrix of the surface material of the medical device); U.S. Pat. No. 4,107,121 (constructing the medical device with ionogenic hydrogels, which thereafter absorb or ionically bind antibiotics); U.S. Pat. No. 5,013,306 (laminating an antibiotic to a polymeric surface layer of a medical device); and U.S. Pat. No. 4,952,419 (applying a film of silicone oil to the surface of an implant and then contacting the silicone film bearing surface with antibiotic powders).

Further, U.S. Pat. Nos. 5,624,704 and 5,902,283 disclose medical devices and methods for impregnating medical implants with antimicrobial agents so that the antimicrobial penetrates the material of the implants. U.S. Pat. Nos. 5,756,145 and 5,853,745 disclose durable antimicrobial coatings for implants, such as orthopedic implants, and methods of coating them. U.S. Pat. No. 5,688,516 describes compositions and methods of employing compositions to flush and coat medical devices, in which the compositions include combinations of a chelating agent, anticoagulant or antithrombotic agent with a non-glycopeptide antimicrobial agent.

B. Combination of Antiobiotics

It is known that a combination of bacteriostatic and bactericidal agents systemically have been ineffective in successfully abrogating the colonization of microbes. Numerous references have cited instances where the combination has actually reduced efficacy of the individual bacteriostatic and bactericidal agents (Rahal (1978), Lepper and Dowling (1951), Strausbaugh and Sande (1978), Rahal et al. (1974), and McCabe et al. (1965)).

The present invention is the first to utilize bacteriostatic and bactericidial antimicrobial compositions in conjunction to reduce putative colonization of a medical device by either coating the medical device or by providing the medium necessary to flush the medical device. It is envisaged that this invention reduces the infection rates related to microbial growth enough that the time a medical device remains implanted inside a patient is increased, thus reducing the medical expenses incurred by patients requiring the medical device. Complications related to growth of biolfim, a by product of excessive microbial proliferation commonly found on the surfaces of medical device, is also potentially reduced from effective flushing of the medical device, thus minimizing microbial related complications.

### BRIEF SUMMARY OF THE INVENTION

The present invention relates to coated medical devices, kits to coat medical devices and methods of coating such medical devices. This invention delineates a novel method wherein a medical device is either flushed or coated with a unique combination of bacteriostatic and bactericidal agents. The combination of bacteriostatic and bactericidal agents reduce, abrogate, or minimize microbial growth and or colonization when compared to uncoated or non-flushed medical devices. Reduction, abrogation, or minimization of microbial growth can be attributed to the combination of the bacteriostatic and bacteriocidal agents acting synergistically and/or additively when used in an effective concentration such that the concentration is effective to reduce the growth of colonization of the microbes by at least 10%, 20%, 30%, 40%, 50%, 60%, 70%, 80%, 90%, 100% or any range therebetween.

An embodiment of the present invention is a method for coating a medical device comprising the steps of applying to at least a portion of the medical device, a bactericidal coating layer, wherein the bactericidal coating layer comprises a bactericidal agent; and applying to at least a portion of the surface of the medical device, a bacteriostatic coating, wherein the bacteriostatic coating layer comprises a bacteriostatic agent wherein the combination of the bactericidal and bacteriostatic agents are in an effective concentration to inhibit growth of microbial organisms relative to an uncoated medical device. The bacteriostatic coating layer and the bactericidal coating layer may be applied simultaneously or consecutively. In other words, the bacteriostatic agent and the bactericidal agents may be combined in the same solution prior to coating the medial device or the agents are applied in layers on the medical device.

In specific embodiments, the bactericidal agent includes, but is not limited to aminoglycosides, penicillins, cephalosporins, carbapenems, glycopeptides, rifamycins, quinolones, fusidic acid, sulfonamides, streptogramins, lipopeptides, and combinations thereof.

Another specific embodiment is that the bacteriostatic agent includes, but is not limited to tetracyclines, macrolides, ketolides, chloramphenicols, oxazolidinones, lincosamindes, and combinations thereof.

A further embodiment is that the medical device is implanted into a subject at risk for infection, wherein the medical device is coated with a composition comprising a bactericidal agent and a bacteriostatic agent.

An embodiment of this invention is that it reduces colonization of gram positive bacteria, gram negative bacteria, fungi, and mycobacterium.

A further embodiment of this invention is that it reduces microbial growth not only on medical devices that are coated, but in flushing both coated and uncoated medical devices.

Another embodiment of this invention is that it has coated on one or more of its surfaces or at least on a portion of the surface an antibiotic composition comprising a combination of an aminoglycoside based drug and a tetracycline based drug, the combination coated is in an amount effective to inhibit microbial growth.

Yet further, another embodiment of the present invention is that the combination of the bactericidal agent and bacteriostatic agent comprises a kit not only used for coating medical devices, but also for flushing the medical devices. In certain embodiments, the kit contains a combination of an aminoglycoside and tetracycline based drug.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description.

### DETAILED DESCRIPTION OF THE INVENTION

It is readily apparent to one skilled in the art that various embodiments and modifications may be made to the invention disclosed in this application without departing from the scope and spirit of the invention.

### I. DEFINITIONS

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “containing”, “including” and “comprising” are interchangeable and one of skill in the art is cognizant that these terms are open ended terms.

The term “microbe(s)” or “microbial organism” as used herein is defined as a microscopic organism such as bacteria, fungi, microscopic algae, protozoa, and viruses unable to be seen by the naked eye.

The term “bactericidal” as used herein is defined as an antimicrobial agent that: (a) is known by those of skill in the art to kill organisms in bacterial suspensions when used in concentrations that are equivalent to the serum concentrations that are clinically achieved in humans during systemic administration of the antimicrobial agent; and (b) is applied according to this invention (via coating or catheter lock/flush solution) to the surfaces of the medical devices in such a way that the total amount of each antimicrobial agent applied to the surfaces of the whole device does not exceed a daily systemic dose of that antimicrobial agent as an antimicrobial agent used to kill microbes.

The term “bacteriostatic” as used herein is defined as an antimicrobial agent that (a) is known by those of skill in the art to inhibit the growth of organisms in bacterial suspensions when used in concentrations that are equivalent to the usual serum concentrations that are achieved in humans during systemic administration of the antimicrobial agent; and (b) is applied according to this invention (via coating or catheter lock/flush solution) to the surfaces of the medical devices in such a way that the total amount of each antimicrobial agent applied to the surfaces of the whole device does not exceed a daily systemic dose of that antimicrobial agent used to inhibit growth but not kill microbes.

The term “antibiotics” as used herein is defined as a substance that inhibits the growth of microorganisms without damage to the host. For example, the antibiotic may inhibit cell wall synthesis, protein synthesis, nucleic acid synthesis, or alter cell membrane function. The classes of antibiotics used may fall under two categories, bactericidal and bacteriostatic. Bactericidal antibiotics include those from the group consisting of aminoglycosides, penicillins, cephalosporins, carbapenems, glycopeptides, rifamycins, quinolones, fusidic acid, sulfonamides, streptogramins, and lipopeptides. Bacteriostatic antibiotics include those from the group consisting of tetracyclines, macrolides, ketolides, chloramphenicols, oxazolidinones, and lincosamindes. In specific embodiments, bactericidal agents include, but are not limited to, kanamycin, gentamicin, tobramycin, netilmicin, sisomicin, amikacin, ampicillin, amoxicillin, cloxacillin, dicloxacillin, ticarcillin, indanyl carbenicillin, azlocillin, mezlocillin, nafcillin, oxacillin, piperacillin, cefazolin, cephalothin, cephapirin, cephradine, cefamandole, cefonicid, cefuroxime, cefmetazole, cefotetan, cefoxitin, cefotaxime, cefoperazone, ceftazidine, ceftizoxime, ceftriaxone, moxalactam, cefepime, cefpirome, cefadroxil, cephalexin, cephradine, cefaclor, cefprozil, cefuroxime, locracarbef, cefdinir, cefditoren, cefixime, cefpodoxime, ceftibuten, cefepelem, cephamasporin, ceftobiprole, aztreonam, imipenem, meropenem, ertapenem, vancomycin, teicoplanin, dalbavancin, telavancin, rifampin, rifabutin, nalidixic acid, fucidins, sulfamethoxazole, sulfadiazine, sulfisoxazole, sulphafurazole, sulfamethoxazole, sulfamethizole, sulfadimidine, sulfacarbamide, sulfadoxine, sulgaguanidine, sulfathalidine, sulfasalazinesulfamylon, mikamycin, virginiamycin, pristinamycin, quinupristin-dalfopristin and daptomycin. In specific embodiments, bacteriostatic agents include, but are not limited to, oxytetracycline, demeclocycline, doxycycline, minocycline, tigecycline, erythromycin, clarithromycin, azithromycin, spiramycin, telithromycin, linezolid, eperezolid, clindamycin, and lincomycin.

The term “coating” as used herein is defined as a layer of material covering a medical device. The coating can be applied to the surface or impregnated within the material of the implant.

The term “effective concentration” means that a sufficient amount of antimicrobial agent is added to decrease, reduce, abrogate, prevent, or inhibit the growth of bacteria and/or fungal organisms. The amount will vary for each compound and upon known factors such as pharmaceutical characteristics; the type of medical device; age, sex, health and weight of the recipient; and the use and length of use. It is within the skilled artisan's ability to relatively easily determine an effective concentration for each compound.

The term “synergism” as used herein is defined as the combined action of two otherwise antagonistic drugs that results in an enhanced antimicrobial activity compared to the use of any one of those drugs individually.

The term “additively” as used herein is defined as the additive antimicrobial effect when two antagonistic drugs are combined, enhancing the effect of the individual drugs in a linear manner when used together.

The term “gram-negative bacteria” or “gram-negative bacterium” as used herein is defined as bacterium which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “medical device” as used herein refers to any material, natural or artificial that is inserted into a mammal. Particular medical devices especially suited for application of the antimicrobial combinations of this invention include, but are not limited to, insertable central venous catheters, dialysis catheters, tunneled central venous catheters, peripheral venous catheters, percutaneously inserted central venous catheters, peripherally inserted central catheters (PICC), arterial catheters, pulmonary artery Swan-Ganz catheters, vascular catheter ports, wound drain tubes, hydrocephalus shunts, peritoneal dialysis catheters, defibrillators, pace-maker systems, artificial urinary sphincters, joint prostheses or replacements, urinary dilators, urinary devices, tissue bonding devices, penile prostheses, hernia mesh, ventricular catheter, ventricular shunts, urinary incontinence devices, bowel incontinence devices, vascular grafts, drug delivery systems (including pumps, generators, tubings, catheters, sensors, etc), fracture fixation devices, nervous system stimulation devices, bilary stents, nephromty catheter, bladder catheter, epidermal catheter, spinal catheter, bioabsorbable polymers, respiratory devices, endotracheal/nasotracheal tubes, tracheotomy devices, urinary stents, vascular dialators, extravascular dialators, vascular stents, extravascular stents, orthopedic implants, heart assist devices, stents, mammary implants, penial implants, dental devices, cannulas, elastomers, hydrogels, feeding tubes, heart valves, and any other medical device used in the medical field. “Medical devices” also include any device which may be inserted or implanted into a human being or other animal, or placed at the insertion or implantation site such as the skin near the insertion or implantation site, and which include at least one surface which is susceptible to colonization by microbes.

The term “subject” as used herein, is taken to mean any mammalian subject to which the composition/medical device is administered. A skilled artisan realizes that a mammalian subject, includes, but is not limited to humans, monkeys, horses, pigs, cows, dogs, cats, rats and mice. In a specific embodiment, the methods of the present invention are employed to treat a human subject. Thus, the subject may or may not be cognizant of their disease state or potential disease state and may or may not be aware that they are need of treatment (therapeutic treatment or prophylactic treatment).

The term “preventing” as used herein, is taken to mean the act of minimizing, inhibiting, impeding, and/or circumventing the growth of microbes, as previously defined, on at least one surface or at least a portion of one surface of an indwelling medical device, those of which are enumerated above.

The term “inhibiting” or “reducing” as used herein, is taken to mean the act of limiting the growth of microbes, as previously defined, on at least one surface or at least a portion of one surface of an indwelling medical device

### II. MEDICAL DEVICES

There is general appreciation within the medical community the need to minimize infections related to indwelling medical devices. This invention describes for the first time the use of a bacteriostatic and bactericidal agent used for coating and/or flushing medical devices. The unique aspect of this invention is centered around the combination of two otherwise antagonistic antibiotic agents that wouldn't otherwise have been surmised to work in conjunction to reduce microbial colonization. This invention reduces infection rates by reducing putative colonization of a medical device by either coating the medical device or by providing the medium necessary to flush the medical device. The reduction in infection increases the time a medical device remains implanted inside a patient, reducing the medical expenses incurred by patients requiring the medical device. Furthermore, the reduction in infection from effective flushing of the medical device prevents microbial related complications.

The combination of bacteriostatic and bactericidal agents reduce, inhibit, abrogate, or minimize microbial growth and or colonization when compared to uncoated or non-flushed medical devices. Reduction, abrogation, or minimization of microbial growth can be attributed to the combination of the bacteriostatic and bacteriocidal agents acting synergistically and/or additively when used in an effective concentration such that the concentration is effective to reduce the growth of colonization of the microbes by at least 10%, 20%, 30%, 40%, 50%, 60%, 70%, 80%, 90%, 100% or any range therebetween.

Exemplary medical devices include, but are not limited to, insertable central venous catheters, dialysis catheters, tunneled central venous catheters, peripheral venous catheters, percutaneously inserted central venous catheters, peripherally inserted central catheters (PICC), arterial catheters, pulmonary artery Swan-Ganz catheters, vascular catheter ports, wound drain tubes, hydrocephalus shunts, peritoneal dialysis catheters, defibrillators, pace-maker systems, artificial urinary sphincters, joint prostheses or replacements, urinary dilators, urinary devices, tissue bonding devices, penile prostheses, hernia mesh, ventricular catheter, ventricular shunts, urinary incontinence devices, bowel incontinence devices, vascular grafts, drug delivery systems (including pumps, generators, tubings, catheters, sensors, etc), fracture fixation devices, nervous system stimulation devices, bilary stents, nephromty catheter, bladder catheter, epidermal catheter, spinal catheter, bioabsorbable polymers, respiratory devices, endotracheal/nasotracheal tubes, tracheotomy devices, urinary stents, vascular dialators, extravascular dialators, vascular stents, extravascular stents, orthopedic implants, heart assist devices, stents, mammary implants, penial implants, dental devices, cannulas, elastomers, hydrogels, feeding tubes, heart valves, and any other medical device used in the medical field. “Medical devices” also include any device which may be inserted or implanted into a human being or other animal, or placed at the insertion or implantation site such as the skin near the insertion or implantation site, and which include at least one surface which is susceptible to colonization by microbes.

A. Coating

The steps involved in the coating the medical device of the present invention comprises applying to at least a portion of the medical device, a bactericidal coating layer, wherein the bactericidal coating layer comprises a bactericidal agent; and applying to at least a portion of the surface of the medical device, a bacteriostatic coating, wherein the bacteriostatic coating layer comprises a bacteriostatic agent wherein the combination of the bactericidal and bacteriostatic agents are in an effective concentration to inhibit growth of microbial organisms relative to an uncoated medical device.

In certain embodiments, coating at least a portion of the medical device, wherein a portion is herein designated as a part, whole, or any designation in between these two boundaries. At least a portion implies coverage of the medical device in such a way that the entire medical device is eventually coated, but since the present invention uses the combination of two disparate antimicrobial agents, one bacteriostatic, the other bactericidal, there is a mixed distribution of the coating/impregnation solution on the surface of the medical device, specific in part to the intended clinical purpose of the medical device, the duration of the medical devices implantation, and other various parameters used in determining the appropriate mixture of bacteriostatic and bactericidal agents so that effective antimicrobial activity is achieved. One of skill in the art is aware that the bacteriostatic agent and the other bactericidal agent can be combined within the same coating layer or they may be applied separately. Thus, one of skill in the art realizes that coating at least a portion of a medical device can include coating at least 1%, at least 10%, at least 20%, at least 30%, at least 40%, at least 50%, at least 60%, at least 70%, at least 80%, at least 90 % or at least 100% of the medical device, or any range there between. Thus, coating a portion of a medical device comprises coating at least 5% to at least a 100% of the device.

As outlined in U.S. Pat. No. 6,475,434, herein included as a reference in its entirety, the medical devices that are amenable to impregnation by the antimicrobial combinations are generally comprised of a non-metallic or metallic material such as thermoplastic or polymeric materials. Examples of such materials are rubber, plastic, polyethylene, polyurethane, silicone, Gortex (polytetrafluoroethylene), Dacron (polyethylene tetraphihalate), polyvinyl chloride, Teflon (polytetrafluoroethylene), latex, elastomers, nylon and Dacron sealed with gelatin, collagen or albumin. Examples of metallic materials include, but are not limited to, tivanium, titanium, and stainless steel.

Bioabsorbable polymers may also be amenable to coating. The bioabsorbable polymers aid in orthopedic situations where the financial and physical cost of surgery to remove a medical device is too high and inconvenient. Some bioabsorbable polymers that could be used, but are not limited, include Polyglycolic acid, Polylactic acid, Polydiaxanone, Polycarpolactone, Polyhydroxybutyrate, Poly-amino acids, and any combinations of these polymers thereof.

The amount of each antimicrobial agent used to coat the medical device varies to some extent, but is at least a sufficient amount to form an effective concentration to inhibit the growth of microbial organisms. The dual combination of the bactericidal and bacteriostatic antimicrobial agents are dispersed through the surface of the medical device. The amount of each antimicrobial agent used to impregnate the medical device varies to some extent, but is in at least in an effective concentration to inhibit the growth of microbial organisms. The antimicrobial agents can be applied to the medical device in a variety of methods. Exemplary application methods include, but are not limited to, spraying, painting, dipping, sponging, atomizing, smearing, impregnating and spreading.

In a preferred embodiment, the step of forming an antimicrobial composition may also include the step of adding an alkalinizing agent to the composition in order to enhance the reactivity of the material of the medical implant, as outlined in U.S. Pat. No. 5,902,283, herein incorporated in its entirety by reference. Further, according to the preferred embodiment, the antimicrobial composition is heated to a temperature between about 30° C. and 70° C. prior to applying the composition to the medical implant to increase the adherence of the antimicrobial agent to the medical implant material. After the impregnated implant is removed from the antimicrobial solution and allowed to dry, the impregnated implant is preferably rinsed with a liquid and milked to remove excess granular deposits and ensure uniform color of the impregnated implant. The antimicrobial composition may be applied to the medical implant by dipping the implant into the antimicrobial composition for a period of between 15 and 120 minutes, and then removing the impregnated implant from the composition. Preferably, the implant is dipped in the composition for a period of approximately 60 minutes.

As noted in U.S. Pat. No. 5,902,283, the method of the present invention preferably comprises a single step of applying both antimicrobial compositions to the surfaces of a medical device. However, it is expected that several applications of the antimicrobial agent can be applied to the surface of the implant without affecting the adherence of the antimicrobial agent. One skilled in the art is cognizant that both antimicrobial agents can be applied together in a single step. Thus, the method of the application of the antimicrobial agents can vary and should not be limited to the described methods. Furthermore, a skilled artisan recognizes that the order of the application of the compositions of both antimicrobial drugs is not relevant and can vary for any given application to a medical device

Another preferred embodiment of the present invention is directed to a medical implant having an antimicrobial layer and a protective layer, and a method for coating such an implant with an antimicrobial layer and a protective layer as delineated in U.S. Pat. No. 5,756,145, herein incorporated in its entirety as a reference. The protective layer slows the leaching of antimicrobial agents from the surface of the implant and is resilient to resist sloughing of the antimicrobial agents during implantation. Further, the protective layer can also protect certain photosensitive antimicrobial agents from exposure to light or air. For instance, some antimicrobial agents, such as methylisothiazolone, are regarded as photosensitive. Moreover, when orthopedic devices coated with a single layer of PV-coVA-co-VA mixed with minocycline and rifampin were exposed to air for few days, the coating layer became dry, got darker in color, and became much more likely to slough off the device upon scratching.

In one aspect of the present invention the protective coating layer, applicable particularly to those orthopedic devices previously outlined, can be a single layer. It is either a durable coating layer or a resilient coating layer. In the preferred embodiment the protective coating layer is at least two layers and includes a durable coating layer and a resilient coating layer.

According to another aspect of the present invention, the protective coating layer is preferably comprised of a durable coating layer, such as a mixture of collodion and nylon and a resilient coating layer such as collodion. The nylon is preferably selected from the group consisting of polycaprolactam, polylauryl-lactam and polyhexamethylene sebacamide. The order of the protective layers can be either with the resilient layer coating the dual bacteriostatic/bactericidal antimicrobial layer and the durable layer coating the resilient layer or the reverse, i.e., the durable layer coating the antimicrobial layer and the resilient layer coating the durable layer. The method for this coating technique is not limited to the examples provided in U.S. Pat. No. 5,756,145.

One method amenable for treating non-metallic medical devices, as outlined in U.S. Pat. No. 6,589,591, is the use of glycerol in the coating process in order to increase efficacy of the adherence of the antimicrobial combination to the medical device. The treatment solution consists of a solvent of a saturated short chain monocarboxylic acid such as formic acid, acetic acid, and propionic acid with a liquidity state below 90° C. and above 10° C. and a pKa of 3 to 5. The formic acid solution is 88% formic acid. It is mixed with an 85% ortho-phosphoric acid solution (for uniformity of coating), 10 mg of potassium chloride per ml of the mixture of formic acid and ortho-phosphoric acid to get a homogeneous solution (potassium ions facilitate surface binding by increasing the ionic strength), and glycerol. The glycerol or glycerin is used as a plasticizer and a vehicle solvent. It also acts as a lubricant between polymer chains to prevent the polymer from becoming brittle during the treatment process. The glycerol also forms hydrogen bonding with its hydroxyl groups [—OH] with the polymer as well as the antimicrobial agents during the treatment process facilitating the incorporation of coating agents (antimicrobial or non-antimicrobial) into the medical device. The total volume of the resulting coating mixture can be composed of 79% formic acid solution (range between 10% to 90%), 8% ortho-phosphoric acid solution (range between 5% to 10%), and 13% glycerin (range between 8% to 15%). The antimicrobial agents are added to the solution before addition of glycerin to avoid dissolution at a higher viscosity that glycerin adds to the coating solution. One skilled in the art recognizes that any combination of the bacteriostatic and bactericidal agents can be used as long as the synergistic effect has been shown to be effective in reducing microbial colonization.

After the treatment period, the device is removed and shaken vigorously or purged with nitrogen gas to remove any excess solution from the device. The device is then placed under a well-ventilated fume hood for at least 16 hours (it is recommended to dry for 48 hours to insure removal of excess glycerin and formic acid). This drying step is optimally performed in the dark. After the drying period the device is rinsed and flushed with deionized water and placed back under the fume hood for another 10-24 hour period.

One skilled in the art recognizes that the myriad techniques employed and designed in the following patents are not limiting, and thus are examples of methods by which one can coat and/or impregnate a medical device for medical applications, included herein in their entirety as references: U.S. Pat. Nos. 6,475,434, 4,442,133, 4,917,686, 4,107,121, 5,013,306, 4,895,566 , 5,624,704 and 4,952,419.

B. Flushing

Another aspect to this invention is the application of the combination of bacteriostatic and bactericidal agents used for flushing catheters and other medical devices.

Microorganisms that attach themselves to inert surfaces, such as those medical devices that have been previously listed, produce a layer made of exopolysaccharide called microbial biofilm. These organisms embed themselves in this layer. This biofilm layer ultimately becomes the protective environment that shields these organisms on the inert surface from the antimicrobial activity of various antibiotics or antiseptics. In U.S. Pat. Nos. 5,362,754 and 5,688,516, incorporated herein by reference in their entirety, it is demonstrated that a combination of one or more antimicrobial agent with one or more chelator and/or anticoagulant (such as EDTA or heparin) reduces or eradicates these antibiotic-resistant biofilm embedded microorganisms if the antimicrobial and chelator combination is allowed to dwell on the surface for anywhere between 15 minutes to 4 hours, if necessary. One skilled in the art readily recognizes that the present invention is a novel departure from previous inventions in that the present invention discloses the specific use of a bacteriostatic agent in synergistic combination with a bactericidal agent.

In other embodiments, biofilm penetrating agents combined with base materials can be used in order to effectively penetrate a biofilm composition successfully (U.S. Pat. No. 6,475,434, herein incorporated in its entirety as reference). Suitable biofilm penetrating agents include the amino acid cysteine and cysteine derivatives. Examples of these agents include cysteine (L-cysteine, D-cysteine, DL-cysteine), DL-Homocysteine, L-cysteine methyl ester, L-cysteine ethyl ester, N-carbamoyl cysteine, cysteamine, N-(2-mercaptoisobutyryl)-L-cysteine, N-(2-mercaptopropionyl)-L-cysteine-A, N-(2-mercaptopropionyl)-L-cysteine-B, N-(3-mercaptopropionyl)-L-cysteine, L-cysteine ethyl ester hydrochloride, nacystelyn (a lysine salt of N-acetylcysteine), N-acetylcysteine, and derivatives thereof. Preferably, the biofilm penetrating agent is N-acetylcysteine and derivatives thereof (U.S. Pat. No. 6,475,434). Other derivatives of N-acetylcysteine, including esters, amides, anhydrides, and thio-esters and thio-esters of the sulfhydryl moeity, can be used as well as biofilm penetrating agents.

It is also contemplated that pharmaceutically acceptable salts of N-acetylcysteine and derivatives of N-acetylcysteine may also be used as biofilm penetrating agents. Non-limiting examples of these salts include sodium salts, e.g., N-acetyl-L-cysteine sodium salt and N-acetyl-L-cysteine sodium zinc monohydrate, potassium salts, magnesium salts, e.g., N-acetyl-L-cysteine magnesium zinc salts, calcium salts, e.g., N-acetyl-L-cysteine calcium zinc monohydrate, zinc salts, e.g., N-acetyl-L-cysteine zinc salt, zinc mercaptide salts, ammonium slats, e.g., N-acetyl-L-cysteine ammonium zinc salt, calcium zinc N-acetyl-L-cysteinate acetate, zinc mercaptide N-acetylcysteine carboxylates, and alkyl ammonium and alkanol ammonium salts, i.e., wherein the ammonium ion is substituted with one or more alkyl or alkanol moieties (U.S. Pat. No. 6,475,434, which is incorporated herein by reference in its entirety).

The biofilm penetrating agent is included in the biofilm penetrating composition in amounts sufficient to penetrate, or break-up the biofilm and provide the biofilm penetrating agent, antimicrobial agent, and/or antifungal agent access to the biofilm embedded microorganisms thereby facilitating the removal of substantially all of the biofilm embedded microorganisms from at least one surface of the medical device. While the biofilm penetrating agent may be 100% of the biofilm penetrating composition, preferably, the biofilm penetrating composition contains from at least about 0.01% to about 60% biofilm penetrating agent by weight based upon the total weight of the biofilm penetrating composition being employed. In the preferred embodiment, the biofilm penetrating composition includes from at least about 0.5% to about 30% (by weight) biofilm penetrating agent (U.S. Pat. No. 6,475,434).

The term “base material” is defined herein as any of a group of materials which effectively disperses the biofilm penetrating agent at an effective concentration to penetrate, or break-up, the biofilm thereby facilitating access of the biofilm penetrating agent, antimicrobial agents, and/or antifungal agents to the microorganisms embedded in the biofilm, and thus, removal of substantially all of the microorganisms from at least one surface of the medical device. The term “base material” also includes any group of solutions which effectively disperse the biofilm penetrating agent at an effective concentration to form a biofilm penetrating composition coating for medical devices which substantially prevents the growth or proliferation of biofilm embedded microorganisms on at least one surface of the medical device. In the case of the biofilm penetrating composition coating, preferably, the base material also facilitates the adhesion of the biofilm penetrating composition to at least one surface of the medical device and prevents the biofilm penetrating composition coating from being easily removed from the surface of the medical device, thereby facilitating the utilization of the biofilm penetrating composition to coat at least one surface of a medical device (U.S. Pat. No. 6,475,434).

Examples of suitable base materials include, but are not limited to, buffer solutions, phosphate buffered saline, saline, water, polyvinyl, polyethylene, polyurethane, polypropylene, silicone (e.g., silicone elastomers and silicone adhesives), polycarboxylic acids, (e.g., polyacrylic acid, polymethacrylic acid, polymaleic acid, poly-(maleic acid monoester), polyaspartic acid, polyglutamic acid, aginic acid or pectimic acid), polycarboxylic acid anhydrides (e.g., polymaleic anhydride, polymethacrylic anhydride or polyacrylic acid anhydride), polyamines, polyamine ions (e.g., polyethylene imine, polyvinylarnine, polylysine, poly-(dialkylamineoethyl methacrylate), poly-(dialkylaminomethyl styrene) or poly-(vinylpyridine)), polyammonium ions (e.g., poly-(2-methacryloxyethyl trialkyl ammonium ion), poly-(vinylbenzyl trialkyl ammonium ions), poly-(N.N.-alkylypyridinium ion) or poly-(dialkyloctamethylene ammonium ion) and polysulfonates (e.g. poly-(vinyl sulfonate) or poly-(styrene sulfonate)), collodion, nylon, rubber, plastic, polyesters, Gortex (polytetrafluoroethylene), Dacron® (polyethylene tetraphthalate), Teflon polytetrafluoroethylene), latex, and derivatives thereof, elastomers and Dacron(® sealed with gelatin, collagen or albumin, cyanoacrylates, methacrylates, papers with porous barrier films, adhesives, e.g., hot melt adhesives, solvent based adhesives, and adhesive hydrogels, fabrics, and crosslinked and non-crosslinked hydrogels, and any other polymeric materials which facilitate dispersion of the biofilm penetrating agent and adhesion of the biofilm penetrating coating to at least one surface of the medical device. Linear copolymers, cross-linked copolymers, graft polymers, and block polymers, containing monomers as constituents of the above exemplified polymers may also be used (U.S. Pat. No. 6,475,434).

While the biofilm penetrating composition may include any number of biofilm penetrating agents and base materials, in the case of internal or external use of the biofilm penetrating composition with humans or animals, the biofilm penetrating agent and base material should be biocompatible with the human beings or animals in which the medical device is inserted or implanted. “Biocompatible” is herein defined as compatible with living tissues, such that the medical device is not rejected or does not cause harm to the living tissue (U.S. Pat. No. 6,475,434).

In clinical situations, it is typically not feasible to allow a 4 hour dwell time for the chelator and antimicrobial agent to reduce or eradicate the microbes. For example, it may not be possible to interrupt the therapy of critically ill patients receiving continuous infusion therapy through a vascular catheter for 4 hours. Thus, a further embodiment of this invention is to use the bacteriostatic/bactericidal combination as a flushing agent (with combinations of at least one chelator/anticoagulant in a preparation in alcohol) that allows rapid reduction and/or eradication of microorganisms embedded in a biofilm in a time as short as about 15 minutes. This is exemplified in U.S. Publication No. 20050013836, which is incorporated herein by reference in its entirety.

A number of exemplary chelating agents, in combination with bacteriostatic/bactericidal agents, can be used in the flushing of a medical device include, but are not limited to, EDTA, EGTA, EDTA 2Na, EDTA 3Na, EDTA 4Na, EDTA 2K, EDTA 2Li, EDTA 2NH4, EDTA 3K, Ba(II)-EDTA, Ca(II)-EDTA, Co(II)-EDTA, Cu(II)-EDTA, Dy(III)-EDTA, Eu(III)-EDTA, Fe(III)-EDTA, In(III)-EDTA, La(III)-EDTA, CyDTA, DHEG, diethylenetriamine penta acetic acid (DTPA), DTPA-OH, EDDA, EDDP, EDDPO, EDTA-OH, EDTPO, EGTA, HBED, HDTA, HIDA, IDA, Methyl-EDTA, NTA, NTP, NTPO, O-Bistren, TTHA, DMSA, deferoxamine, dimercaprol, zinc citrate, a combination of bismuth and citrate, penicillamine, succimer or Etidronate. Other chelating agents not listed here, but that serve the similar function of binding barium, calcium, cerium, cobalt, copper, iron, magnesium, manganese, nickel, strontium, or zinc will be acceptable for use in this aspect of the invention (See U.S. Publication No. 20050013836).

In further embodiments, it is contemplated that the lock-in solution used to flush medical devices contains an effective concentration of bacteriostatic and bactericidal agents that act in synergy in order to enhance the efficacy of the lock-in solution. The flushing solution of the present invention may or may not require an anticoagulant and/or a chelator. It is further contemplated that the flushing solution of the present invention can be left to wash over the medical device between 15 minutes to 4 hours, or any time between in order for the solution to effectively eliminate further colonization and to break up established biofilm layers on the medical device. It is envisaged that the unique combination of a bacteriostatic and bactericidal agents in this solution can in fact be more effective in preventing, abrogating, and reducing microbial colonization on medical devices than a single agent alone.

### III. KITS

Another embodiment of this invention is a kit comprising compositions to coat or flush the surfaces of medical devices prior to implantation into a mammal comprising two different antimicrobial agents, a bacteriostatic and a bactericidal. The kit will be packaged for commercial use of coating medical devices or it will be contained as a package for flushing

A further embodiment of this invention is a kit comprising of a solution containing the bactericidal and bacteriostatic agents in an effective concentration to reduce colonization of microbial organisms when used to coat and/or flush medical devices. Described herein are various packaging techniques that may be employed in providing the flush solutions of the invention as part of a commercially available kit, a detailed description provided in U.S. Publication No. 20050013836A. The kit will optionally include an instruction sheet insert to identify how the kit is to be used.

The kits described in this section are exemplified by a solution comprising of a bacteriostatic and bactericidal agent, preferably a tetracycline and an aminoglycoside based drug, as the antibiotic, EDTA as the chelator/anticoagulant, and ethanol. However, as will be appreciated by the skilled artisan, any other combination of one or more antibiotic, one or more chelator/anticoagulant, and ethanol as described in the present disclosure may be packaged in a similar manner. The kit may comprise of one or two or three or more compartments. The components of the kit may be provided in separate compartments or in the same compartment. The components of the kit may be provided separately or mixed. The mixed components may contain two or more agents such as an antibiotic, a chelator/anticoagulant, or ethanol, or additional component.

One of the packaging options below maintain the ingredients, for example, the antibiotic and the chelating agent/anticoagulant, for example EDTA, in an uncombined form. These components are to be combined shortly before use. These packaging options are contemplated to be part of a 2-compartment or three-compartment container system to provide a total volume of about 3 ml of the ready to use preparation. Any compartmentalized container system may be used to package the compositions of the present invention. The options outlined below are envisaged to be non-limiting examples of how the lock/flush solution described herein can be packaged, compartmentalized, and commercialized.

The various compartmentalized embodiments of the present invention as disclosed above, may be provided in a kit form. Such kits would include a container means comprising a volume of diluent, comprising an alcohol optionally diluted if required in a solution such as saline or sterile water, a second (or more) container means comprising one or more antimicrobial or biocide, a third (or more) container means comprising one or more chelating/anticoagulant agent. The dry components may optionally be mixed in one compartment. The addition of the diluent would then be performed immediately prior to use.

The container means of the kits will generally include at least one vial, test tube, flask, bottle, syringe or other container means, into which the antimicrobial/chelator/anticoagulant/alcohol may be placed, and preferably, suitably aliquoted. Where a second or third antibiotic agent, other chelator, alcohol, or additional component is provided, the kit will also generally contain a second, third or other additional container into which this component may be placed. The kits of the present invention will also typically include a means for containing the alcohol, antimicrobial agent, chelator/anticoagulant, and any other reagent containers in close confinement for commercial sale. Such containers may include injection or blow-molded plastic, or glass containers into which the desired vials are retained.

### IV. INHIBITION OF MICROBIAL GROWTH

The present invention utilizes a combination of typically considered antagonistic agents, bacteriostatic and bactericidal agents, to achieve inhibition of microbial growth or colonization. The inhibition can be synergistically or additively. The references included herein in their entirety, Rahal (1978), Lepper and Dowling (1951), Strausbaugh and Sande (1978), Rahal et al. (1974), and McCabe et al. (1965), describe the apparent antagonisms between certain bacteriostatic and bactericidal drugs when used in combination systemically. Despite these apparent published antagonisms, the present invention demonstrates a unique pharmaceutical combination of the two disparate antimicrobial agents on medical devices that, when used in an effective concentration, reduce microbial colonization.

In an exemplary embodiment of the present invention, the combination of antimicrobial agents comprise an aminoglycoside based drug in combination with a tetracycline based drug. In this case, the combination of minocycline (a tetracycline), which is very active against both methicillin-sensitive and methicillin-resistant staphylococci and possess some activity against gram-negative bacteria, with tobramycin (an aminoglycoside) effectively reduces the growth of both gram-positive and gram-negative bacteria in vitro. Thus, this combination may effectively reduce almost all gram-negative bacteria.

Furthermore, this unique combination of a bactericidal agent with a bacteriostatic agent is more effective at bacterial reduction than when a single bactericidal or bacteriostatic agent is solely used topically. This unique combination is further demonstrated by the theoretically antagonistic interaction between bacteriostatic and bactericidal agents when given through an oral administration or systemically. The mechanisms of action for both bactericidal and bacteriostatic drugs are antagonistic since both bind to the 30S subunit of ribosomes in order to eliminate the pathogen. Solubility antagonisms exist as well, since aminoglycosides are very soluble in water but not in organic solvents, while tetracyclines are very soluble in organic solvents; in this invention, however, both are successfully dissolved in an organic acid, formic acid. There are antagonisms in their antimicrobial activities as well, where tetracyclines bind to calcium while aminoglycosides displace magnesium and calcium bridges that link adjoining LPS molecules. In general, aminoglycosides have not empirically been used for prevention of device-related infections, while tetracyclines have been shown to systemically reduce the antimicrobial activity of aminoglycosides. The synergism between these two classes is inconceivable for all the aforementioned reasons, yet in the unique application of the present invention, there is marked enhanced antimicrobial effect as shown below in the examples.

Other bactericidal-bacteriostatic combinations that can be used in the present invention include (a) aminoglycosides-Sulfonamides (includes sulfadiazine, sulfisoxazole, sulphafurazole, sulfamethoxazole, sulfamethizole, sulfadimidine, sulfacarbamide, sulfadoxine, sulgaguanidine, sulfathalidine, sulfasalazinesulfamylon) and (b) aminoglycosides-trimethoprim and/or (c) aminoglycosides-clindomycin/lincocomycin

Yet further, other bactericidal agents that can be used in the present include those from the group consisting of aminoglycosides, penicillins, cephalosporins, carbapenems, glycopeptides, rifamycins, fusidic acid, sulfonamides, streptogramins, and lipopeptides. Bacteriostatic antibiotics include those from the group consisting of tetracyclines, macrolides, ketolides, oxazolidinones, and lincosamindes. More specifically, bactericidal agents include, but are not limited to, kanamycin, gentamicin, tobramycin, netilmicin, sisomicin, amikacin, ampicillin, amoxicillin, cloxacillin, dicloxacillin, ticarcillin, indanyl carbenicillin, azlocillin, mezlocillin, nafcillin, oxacillin, piperacillin, cefazolin, cephalothin, cephapirin, cephradine, cefamandole, cefonicid, cefuroxime, cefmetazole, cefotetan, cefoxitin, cefotaxime, cefoperazone, ceftazidine, ceftizoxime, ceftriaxone, moxalactam, cefepime, cefpirome, cefadroxil, cephalexin, cephradine, cefaclor, cefprozil, cefuroxime, locracarbef, cefdinir, cefditoren, cefixime, cefpodoxime, ceftibuten, cefepelem, ceftobiprole, cephamasporin, aztreonam, imipenem, meropenem, ertapenem, vancomycin, teicoplanin, dalbavancin, or telavancin, rifampin, rifabutin, nalidixic acid, fucidins, sulfamethoxazole, sulfadiazine, sulfisoxazole, sulphafurazole, sulfamethoxazole, sulfamethizole, sulfadimidine, sulfacarbamide, sulfadoxine, sulgaguanidine, sulfathalidine, sulfasalazinesulfamylon mikamycin, virginiamycin, pristinamycin, quinupristin-dalfopristin and daptomycin.

Other bacteriostatic agents that can be used in the present invention include, but are not limited to, oxytetracycline, demeclocycline, doxycycline, minocycline, or tigecycline. In specific embodiments, bacteriostatic agents include, but are not limited to, erythromycin, clarithromycin, azithromycin, spiramycin, telithromycin, chloramphenicol, linezolid, eperezolid, clindamycin, and lincomycin.

Thus, the present invention is utilized to markedly inhibit, reduce, prevent, abrogate, or minimize bacterial colonization by coating medical devices with a bacteriostatic and bactericidal agent, or to flush the medical device in order to achieve the latter stated results. Reduction, abrogation, minimization or prevention of microbial growth can be attributed to the combination of the bacteriostatic and bacteriocidal agents acting synergistically and/or additively when used in an effective concentration such that the concentration is effective to reduce the growth of colonization of the microbes by at least 10%, 20%, 30%, 40%, 50%, 60%, 70%, 80%, 90%, 100% or any range therebetween.

Those bacteria that may be susceptible to the antimicrobial compositions include, but are not limited to, gram positive and gram negative bacteria. Gram-negative bacteria, classified by the Gram stain as having a red stain, have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species. Gram-positive bacteria, classified using the Gram stain as having a blue stain, have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Antibiotic Combinations Synergistically Inhibit Bacterial Growth in a Hernia Patch

The device used in this example is a composite hernia patch with polypropylene mesh on one side and polytetrafluoroethylene (PTFE) on the other side. Antibiotics were incorporated onto devices by using a patented method (U.S. Pat. No. 6,589,591) that utilized coating solutions that contained 100 mg/ml of minocycline; 100 mg/ml tobramycin; 100 mg/ml gentamicin; 100 mg/ml minocycline and 100 mg/ml tobramycin (minocycline dissolved first); or 100 mg/ml minocycline and 100 mg/ml gentamicin (minocycline dissolved first). The tested square device segments were 10 mm (long)×10 mm (wide)×2 mm (thick). The device segments were placed onto agar with their long axis perpendicular to the agar (i.e. only 2 mm of the segment was in contact with the agar). All zone of inhibition are expressed in mm, and table 1 summarizes the results of the zones of inhibition.

TABLE 1

| | Enterococcus | Staphyulococcus | |
|---|---|---|---|
| | faecalis | aureus | Escherichia coli |
| Treatment | (gram positive) | (gram positive) | (gram negative) |
| Minocycline | 3 | 11 | 3 |
| Tobramycin | 9 | 12 | 18 |
| Gentamicin | 0 | 14 | 11 |
| Minocycline & | 14 | 14 | 20 |
| Tobramycin | | | |
| Minocycline & | 8 | 19 | 21 |
| Gentamicin | | | |

Alone, the maximum zone of inhibition of minocycline, tobramycin, or gentamicin are not nearly as effective as when they are combined together against both gram positive and gram negative bacteria. These results show that the combination of a tetracycline based drug (bacteriostatic) with an aminoglycoside based drug (bactericidal) is more effective than a single drug-based therapy alone. Furthermore, albeit there are similar zones of inhibition between gentamicin alone and minocycline & tobramycin, note the dual efficacy seen in both the gram positive and gram negative strains of bacteria. This will prove invaluable in a clinical setting where these medical devices can be exposed to both types of bacteria.

### Example 2

### Antibiotic Combinations Synergistically inhibit Bacterial Growth in a Venous Catheter

The device used in this example is a 7-french, polyurethane central venous catheter. Antibiotics were incorporated onto devices by using a patented method (U.S. Pat. No. 6,589,591) that utilized coating solutions that contained 100 mg/ml of minocycline; 100 mg/ml tobramycin; 100 mg/ml gentamicin; 100 mg/ml minocycline and 100 mg/ml tobramycin (minocycline dissolved first); or 100 mg/ml minocycline and 100 mg/ml gentamicin (minocycline dissolved first). The tested catheter segments were 10 mm (long)×2 mm (wide). The cather segments were placed onto agar with their long axis parerrel to the agar. All zone of inhibition are expressed in mm, and table 2 summarizes the results of the zones of inhibition.

TABLE 2

| Treatment | Staphylococcus epidermidis |
|---|---|
| Minocycline | 30 |
| Tobramycin | 14 |
| Gentamicin | 6 |
| Minocycline & Tobramycin | 30 |
| Minocycline & Gentamicin | 30 |

These data show a number of key elements to the combination of these disparate antimicrobial agents. The first is that the antimicrobial agents were successfully incorporated onto a different device besides the previous example, demonstrating the versatility of the application on different medical devices. Secondly, this further establishes the antimicrobial action of these drugs, since there have been previous reports in the literature of antagonisms in the mechanisms of action.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 4,107,121
U.S. Pat. No. 4,442,133
U.S. Pat. No. 4,895,566
U.S. Pat. No. 4,917,686
U.S. Pat. No. 4,952,419
U.S. Pat. No. 5,013,306
U.S. Pat. No. 5,624,704
U.S. Pat. No. 5,688,516
U.S. Pat. No. 5,756,145
U.S. Pat. No. 5,902,283
U.S. Pat. No. 6,475,434
U.S. Pat. No. 6,719,991
U.S. Publication No. 20050013836
Rahal, James J. Medicine, Vol. 57(2): 179-195 (1978).
Lepper, Mark H. & Dowling, Harry F. A.M.A. Archives of Internal Medicine, Vol. 88(4):489-94(1951).
Solomon, D. D. and Sherertz, R. J., J. Controlled Release, 6:343-352 (1987)
Strausbaugh, Larry K. & Sande, Merle A. The Journal of Infectious Diseases, Vol.137(2): 251-260 (1978).
Rahal, James J. et al. New England Journal Of Medicine, Vol. 290 (25): 1394-1398 (1974).
McCabe, William R & Jackson, George G. New England Journal of Medicine, Vol. 272(20): 137-44 (1965).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for coating a medical device comprising the steps of:
applying to at least a portion of the surface of the medical device, a bactericidal coating layer, wherein the bactericidal coating layer comprises a bactericidal agent; and
applying to at least a portion of the surface of the medical device, a bacteriostatic coating, wherein the bacteriostatic coating layer comprises a bacteriostatic agent wherein the combination of the bactericidal and bacteriostatic agents are in an effective concentration to reduce the growth of microbial organisms relative to an uncoated medical device.

2. The method of claim 1, wherein the bactericidal agent and the bacteriostatic agent act to reduce the colonization of microbes.

3. The method of claim 1, wherein the microbial organisms are selected from the group consisting of gram positive bacteria, gram negative bacteria, fungi, mycobacterium and a combination thereof.

4. The method of claim 1, wherein the medical device is selected from the group consisting of insertable central venous catheter, dialysis catheter, tunneled central venous catheter, peripheral venous catheter, percutaneously inserted central venous catheter, peripherally inserted central catheter (PICC), arterial catheter, pulmonary artery Swan-Ganz catheter, vascular catheter port, wound drain tube, hydrocephalus shunt, peritoneal dialysis catheter, defibrillator, pace-maker system, artificial urinary sphincter, joint prosthese or replacement, urinary dilator, urinary device, tissue bonding device, penile prosthese, hernia mesh, ventricular catheter, ventricular shunt, urinary incontinence device, bowel incontinence device, vascular graft, drug delivery system, fracture fixation device, nervous system stimulation device, bilary stent, nephromty catheter, bladder catheter, epidermal catheter, spinal catheter, bioabsorbable polymer, respiratory device, endotracheal/nasotracheal tube, tracheotomy device, urinary stent, vascular dialator, extravascular dialator, vascular stent, extravascular stent, orthopedic implant, heart assist device, mammary implant, penial implant, dental device, cannula, elastomer, hydrogel, feeding tube, heart valve, and a combination thereof.

5. The method of claim 1, wherein the bactericidal agent is selected from the group consisting of aminoglycoside, penicillin, cephalosporin, carbapenem, glycopeptide, rifamycin, quinolone, fusidic acid, sulfonamide, streptogramin, lipopeptide, and a combination thereof.

6. The method of claim 1, wherein the bacteriostatic agent is selected from the group consisting of tetracycline, macrolide, ketolide, chloramphenicol, oxazolidinone, lincosaminde, and a combination thereof.

7. The method of claim 5, wherein the aminoglycosides are selected from the group consisting of kanamycin, gentamicin, tobramycin, netilmicin, sisomicin, amikacin, and a combination thereof.

8. The method of claim 5, wherein the penicillins are selected from the group consisting of ampicillin, amoxicillin, cloxacillin, dicloxacillin, ticarcillin, indanyl carbenicillin, azlocillin, mezlocillin, nafcillin, oxacillin, piperacillin and a combination thereof.

9. The method of claim 5, wherein the cephalosporin is selected from the group consisting of cefazolin, cephalothin, cephapirin, cephradine, cefamandole, cefonicid, cefuroxime, cefmetazole, cefotetan, cefoxitin, cefotaxime, cefoperazone, ceftazidine, ceftizoxime, ceftriaxone, moxalactam, cefepime, cefpirome, cefadroxil, cephalexin, cephradine, cefaclor, cefprozil, cefuroxime, locracarbef, cefdinir, cefditoren, cefixime, cefpodoxime, ceftibuten, cefepelem, cephamasporin, ceftobiprole and a combination thereof.

10. The method of claim 5, wherein the carbapenem is selected from the group consisting of aztreonam, imipenem, meropenem, ertapenem and a combination thereof.

11. The method of claim 5, wherein the glycopeptide is selected from the group consisting of vancomycin, teicoplanin, dalbavancin, telavancin and a combination thereof.

12. The method of claim 5, wherein the rifamycin is rifampin or rifabutin.

13. The method of claim 5, wherein the fusidic acid is fucidin.

14. The method of claim 5, wherein the sulfonamide is selected from the group consisting of sulfamethoxazole, sulfadiazine, sulfisoxazole, sulphafurazole, sulfamethoxazole, sulfamethizole, sulfadimidine, sulfacarbamide, sulfadoxine, sulgaguanidine, sulfathalidine, sulfasalazinesulfamylon and a combination thereof.

15. The method of claim 5, wherein the streptogramin is selected from the group consisting of mikamycin, virginiamycin, pristinamycin, quinupristin-dalfopristin and a combination thereof.

16. The method of claim 5, wherein the lipopeptide is daptomycin,

17. The method of claim 6, wherein the tetracycline is selected from the group consisting of oxytetracycline, demeclocycline, doxycycline, minocycline, tigecycline and a combination thereof.

18. The method of claim 6, wherein the macrolide is selected from the group consisting of erythromycin, clarithromycin, azithromycin, spiramycin and a combination thereof.

19. The method of claim 6, wherein the ketolide is telithromycin.

20. The method of claim 6, wherein the oxazolidinones is linezolid or eperezolid.

21. The method of claim 6, wherein the lincosaminde is clindamycin and lincomycin.

22. A method for treating a subject having an implantable medical device at risk for microbial infections comprising the steps of:
obtaining the medical device as defined in claim 1; and
implanting the medical device into the subject.

23. A method for inhibiting microbial growth on surfaces of an implantable medical device comprising a bactericidal and a bacteriostatic agent wherein the bactericidal agent and bacteriostatic agent are applied as defined in claim 1.

24. An implantable medical device having one or more of its surfaces coated with an antibiotic composition comprising a combination of an aminglycoside and tetracycline, the combination coated in an amount effective to inhibit the growth of microbes.

25. A kit for coating or flushing medical devices, comprising a combination of a bactericidal agent and a bacteriostatic agent in a concentration effective to reduce microbial colonization in a medical device.

26. The kit of claim 25, wherein the bactericidal agent and the bacteriostatic agent are in the same container.

27. The kit of claim 25, wherein the bactericidal agent and the bacteriostatic agent are in different containers.

28. The kit of claim 25, wherein the bactericidal agent is selected from the group consisting of aminoglycoside, penicillin, cephalosporin, betalactam, glycopeptide, rifamycin, fusidic acid, sulfonamide, streptogramin, lipopeptide, and a combination thereof.

29. The kit of claim 25, wherein the bacteriostatic agent is selected from the group consisting of tetracycline, macrolide, ketolide, oxazolidinone, and a combination thereof.

30. A kit for coating the surfaces of medical devices prior to implantation into a subject comprising a combination of aminoglycoside and tetracycline, the combination coated in an amount effective to inhibit the growth of microbial organisms.

31. A method of flushing a medical device comprising the steps of exposing the medical device to solution containing a combination of a bacteriostatic agent and a bactericidal agent in an effective concentration to reduce the colonization of microbes on the surface of the medical device.
